(19) United States
(12) Patent Application Publication
(10) Pub. No.: US 2007/0250116 A1
(43) Pub. Date: Oct. 25, 2007

(54) Tissue Approximator and Retractor Assistive Device

(75) Inventor: Gottumukkala S. Raju, League City, TX (US)

Correspondence Address: LOCKE LIDDELL & SAPP LLP;ATTN: IP DOCKETING, 600 TRAVIS, 3400 CHASE TOWER, HOUSTON, TX, 77002-3095, US

(73) Assignee: BOARD OF REGENTS, THE UNIVERSITY OF TEXAS SYSTEM, Austin, TX (US)

(21) Appl. No.: 11/739,243

(22) Filed: Apr. 24, 2007

Related U.S. Application Data
(60) Provisional application No. 60/794,607, filed on Apr. 25, 2006.

(51) Int. Cl. A61B 17/08

(52) U.S. Cl. 606/216

(57) ABSTRACT

The present disclosure provides an improved method and device for approximation and retraction of tissue portions during a medical procedure. The disclosure includes a tissue approximator-retractor elastic device, comprising: an elastic portion having a contracted length and adapted to expand to a different length to exert a contractile force; and at least three coupling rings spaced along the elastic portion for an operator to choose between the rings and couple at least two rings to at least two tissue portions to adjust a contractile force on the two tissue portions. The disclosure also discloses providing the device near the tissue portions; coupling a first coupling ring to a first tissue portion; choosing between the remaining coupling rings to select a second coupling ring; engaging the second coupling ring and elastically pulling the elastic portion to the second tissue portion to attach to the second tissue portion.

### CROSS REFERENCE TO RELATED APPLICATIONS

This application claims the benefit of U.S. Provisional Application Ser. No. 60/794,607, filed Apr. 25, 2006, which is incorporated herein by reference in its entirety.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO APPENDIX

Not applicable.

### BACKGROUND

1. Field of the Invention

The present disclosure medical procedures. More particularly, the disclosure relates to tissue approximation and retraction for medical procedures.

2. Description of Related Art

During medical procedures on tissues surfaces, it is often desirable to pull tissue portions toward each other, generally known as “approximation”. For example, the tissue portions can be adjacent to a wound that needs suturing or other closure. The gap between adjacent sides of the wound may be so large as to make the closure tenuous and time consuming. Other medical procedures often benefit from pulling tissue portions away from each other, known as “retraction.” The tissue surfaces may benefit from such retraction for medical diagnosis, enhanced viewing and placement during surgical removal, and other medical procedures.

Known medical instruments, such as surgical hemostats and dressing forceps are routinely used for such procedures external to a body of an organism. However, the current endoscopic, laparoscopic, and other medical procedures performed internally to the body create an order of magnitude in complexity. The limited viewing and access during these internal procedures typically prohibit the use of such known instruments for tissue approximation and retraction. The size of the typical instruments is prohibitive for the internal procedures, but more importantly most procedures need to be done with one instrument inserted into the area. One instrument must be able to accomplish multiple tasks often simultaneously. Such limitations constrain the ability to perform tasks that would be simple external to the body. For example, in excising a tumor from an abdominal wall, the tissue to be excised may need retraction from the adjacent tissue to assist proper removal during the excision. Holding a tissue portion away from the adjacent tissue while performing the excision simultaneously with the same instrument is not presently a task that can be done.

Similarly, when closing a gap after an excision or created by a wound, an internally inserted and actuated surgical clip can close a relatively small gap through its known mechanisms by the same instrument inserted into the area. However, a gap that exceeds the capabilities of the clips needs special and time consuming efforts to close. One known method is to start at one end of the gap and slowly close small sections progressively to the other end of the gap with a large number of clips, similar to the operation of a zipper. One recent procedure required about five hours to excise and close a 5 cm tumor.

U.S. Pat. No. 5,972,022 discloses a rubber band with hooks on each end that can be inserted internally into the area needing the medical procedure. The disclosure teaches that the device is inserted into the area, and hooked to two tissue portions on each end of the device. However, the rubber band and hooks are limited to a certain range of movement. Gaps in wounds and excised areas vary. If the rubber band is inserted and proves too long or too short, then it may need removal and another device with a different length inserted. If the rubber band length is only marginally suitable, it can still be used but with perhaps compromised approximation or retraction. Further, the rubber band device does not appear to have any mechanism or method for adjusting the approximation or retraction, or angle of such, during the procedure, especially while the rubber band ends are secured to the tissue portions. For example, the tension force on the tissue portions would vary during an excision, as one tissue portion is excised from another tissue portion. Thus, the device may be useful, but is limited.

Thus, there remains a need for an improved method and device to approximate and retract tissue portions.

### BRIEF SUMMARY

In this field, special and sometimes simple devices from the viewpoint of hindsight can yield major improvements in costs, time, or the ability to even perform a desired medical procedure. The present disclosure provides an improved method and device for better approximation and retraction of tissue portions during a medical procedure. It is believed that the above example of excising and closing a 5 cm tumor in five hours can be reduced to about half the time with the present invention.

The disclosure also provides a method of adjusting a location of a first tissue portion relative to a second tissue portion in a medical procedure, comprising: providing a tissue approximator-retractor elastic device near the tissue portions, the device having an elastic portion with at least three coupling rings spaced along the elastic portion; coupling a first coupling ring to the first tissue portion; choosing between the remaining coupling rings to select a second coupling ring; engaging the second coupling ring and elastically pulling the elastic portion to the second tissue portion; coupling the second coupling ring to the second tissue portion; and allowing the elastic portion of the tissue approximator-retractor elastic device to contract to adjust the location of the first tissue portion relative to the second tissue portion.

The disclosure provides a tissue approximator-retractor elastic device, comprising: an elastic portion having a contracted length and adapted to expand to a different length to exert a contractile force; and at least three coupling rings spaced along the elastic portion and adapted to allow an operator to choose between the rings to couple at least two rings to at least two tissue portions and adjust a contractile force exerted by the elastic portion on the two tissue portions.

### BRIEF DESCRIPTION OF THE DRAWINGS

While the concepts provided herein are susceptible to various modifications and alternative forms, only a few specific embodiments have been shown by way of example in the drawings and are described in detail below. The figures and detailed descriptions of these specific embodiments are not intended to limit the breadth or scope of the concepts or the appended claims in any manner. Rather, the figures and detailed written descriptions are provided to illustrate the concepts to a person of ordinary skill in the art as required by 35 U.S.C. § 112.

FIG. 1 is a schematic diagram of a tissue approximator-retractor elastic device.

FIG. 2 is schematic diagram of another embodiment of the tissue approximator-retractor elastic device.

FIG. 3 is a schematic diagram of exemplary tissue portions separated by a gap.

FIG. 4 is a schematic diagram of the tissue portions with the tissue approximator-retractor elastic device of FIG. 1.

FIG. 5 is a schematic diagram of the tissue portions separated by the gap with at least two tissue engagement members coupled to the tissue approximator-retractor elastic device.

FIG. 6 is a schematic diagram of the tissue portions separated by the gap with the tissue approximator-retractor elastic device anchored on either side of the gap.

FIG. 7 is a schematic diagram of the tissue portions that have been approximated using the tissue approximator-retractor elastic device.

FIG. 8 is a schematic diagram of an exemplary closure of the gap between the tissue portions.

FIG. 9 is a cross-sectional schematic diagram of tissue having a portion to be excised.

FIG. 10 is a cross-sectional schematic diagram of the tissue of FIG. 9 with a tissue approximator-retractor elastic device coupled thereto.

FIG. 11 is a schematic cross-sectional diagram of the first tissue portion partially excised illustrating a retraction using the tissue approximator-retractor elastic device.

FIG. 12 is a cross-sectional schematic diagram of FIG. 11 illustrating a different coupling of a different ring to the tissue portion compared to the tissue approximator-retractor elastic device.

FIG. 13 is a perspective three dimensional diagram illustrating an option to vary an angle of the contractile force between tissue portions.

FIG. 14 is a cross-sectional perspective diagram of the tissue portions to be excised with a plurality of tissue approximator-retractor elastic devices coupled thereto.

### DETAILED DESCRIPTION

One or more illustrative embodiments of the concepts disclosed herein are presented below. Not all features of an actual implementation are described or shown in this application for the sake of clarity. It is understood that the development of an actual embodiment, numerous implementation-specific decisions must be made to achieve the developer's goals, such as compliance with system-related, business-related and other constraints, which vary by implementation and from time to time. While a developer's efforts might be complex and time-consuming, such efforts would be, nevertheless, a routine undertaking for those of ordinary skill in the art having benefit of this disclosure.

In general, the devices and methods herein provide tissue approximation and retraction that can quickly and easily be assembled, positioned, and repositioned in multiple planes and axes of motion. The devices and methods can present relatively little obstruction to the surgeon's movement, protect the sterile field, diminish the risk of tissue trauma, and yet be stable enough to function adequately while reducing the need for assistance.

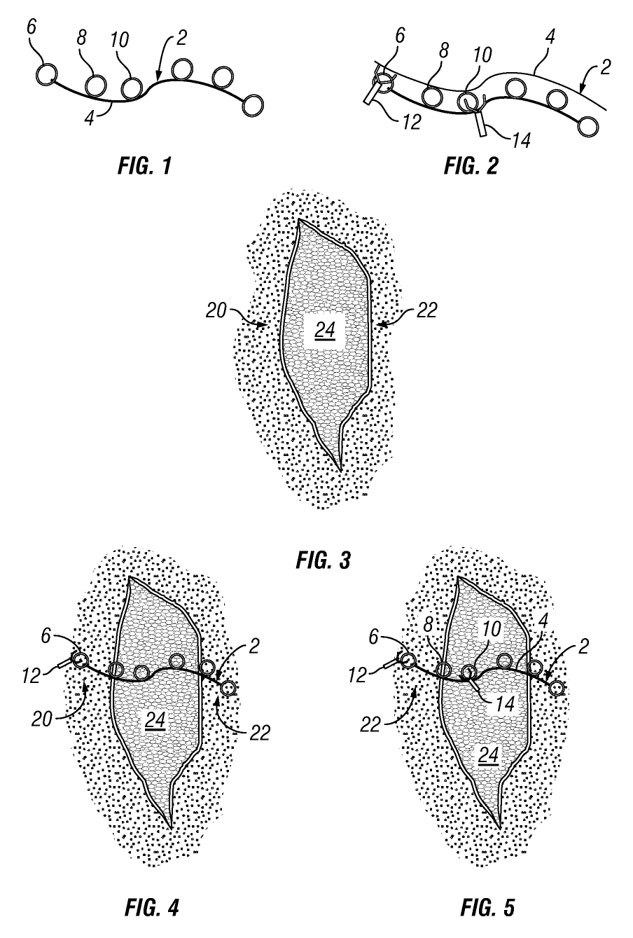

FIG. 1 is a schematic diagram of a tissue approximator-retractor elastic device. The tissue approximator-retractor elastic device 2 generally includes an elastic portion 4 with at least three or more coupling rings 6, 8, 10 spaced along the elastic portion. In at least one embodiment and without limitation, the elastic portion can be approximately 1 mm to 5 mm in diameter and the coupling rings can be about 3 mm in diameter. The length of the elastic portion 4 can vary as may be appropriate and generally would range between 1 cm to 10 cm. However, a key feature is that at least three coupling rings and generally four or more coupling rings are coupled to the elastic portion. The term “ring” is used broadly and includes a full or partial encircling element of any shape, such as circular, square, elliptical, segmented, trapezoidal, and so forth in two or three dimensions. The ring material can be plastic, rubber, metal, ceramic, magnetic, or other medically acceptable materials. The coupling ring 6 can be coupled to the elastic portion 4 on one end, the coupling ring 8 can be attached adjacent to the coupling ring 6, and the coupling ring 10 can be attached adjacent to the ring 8. Other rings can also be coupled to the elastic portion 4. In at least one embodiment, the coupling rings are spaced 0.5 cm from each other along the elastic portion. The elastic portion can be of any suitable elastomeric material, including surgical tubing, non-latex rubber tubing, nitriles, surgical grade rubbers, silicon, and other elastic materials.

The present embodiment provides a significant amount of flexibility during the medical procedure to choose different rings to apply different contractile forces using the same elastic device 2. In other words, the elastic device 2 can be anchored with a first ring, for example, to a tissue portion. After anchoring the first ring, a second ring, selected at the option of the medical personnel, can be pulled to a different tissue portion and anchored thereto. The contractile force can be varied by which ring is selected. Further, the distance between the anchor points can vary not only upon initial setting, but can also be varied during the procedure by selecting other rings during the procedure, as may be beneficial to the particular circumstances. Details are provided below

FIG. 2 is schematic diagram of another embodiment of the tissue approximator-retractor elastic device. The tissue approximator-retractor elastic device 2 can include an elastic portion 4 in which the coupling rings 6, 8, and 10 and other rings are at least partially embedded therein. Thus, the rings form apertures or openings within the material of the elastic portion 4.

One or more tissue engagement members 12, 14 are also shown in FIG. 2. The tissue engagement members 12, 14 are broadly construed to include a variety of devices that can couple the elastic device 2 to a tissue portion. Generally, such engagement members include “clips” as are known to those of ordinary skill in the art for attaching to tissue portions or closing tissue portion in close proximity to each other. The clips generally can be placed and actuated by a single instrument inserted into the area in which the medical procedure will be performed. Sutures can also be used. Staples and tissue adhesives can also be used. In general and as further described below, the engagement member 12 can anchor one portion of the elastic device 2. The second engagement member 14 can engage one of the other rings at the discretion of the operator, such as ring 10, to stretch or pull a portion of the elastic device 2 to a different tissue portion and be coupled thereto. The contractile force caused by the stretched portion is used in a variety of manners to approximate or retract tissues portions.

FIGS. 3-8 are schematic diagrams illustrating an exemplary and nonlimiting procedure using the tissue approximator-retractor elastic device. The figures will be described in conjunction with each other.

FIG. 3 is a schematic diagram of exemplary tissue portions separated by a gap. The tissue portions can represent a number of tissues encountered by medical personnel on or in a body. Thus, “tissue” is used broadly to include organ tissues, such as uterine, esophageal, intestinal, heart, lung, liver, stomach; connective tissues such as ligaments, tendons, cartilage; muscle tissues such as skeletal, smooth, cardiac, and others; dermal tissues; and the like. For example and without limitation, the tissue portions could be tissues separated by a wound which needs closure, or a surgical incision made for the purpose of carrying out a surgical procedure. In some circumstances, the gap 24 is larger than a typical clip inserted into the area can close. Thus, a pre-closure procedure is generally beneficial to allow a more permanent suturing or other closure. The tissue approximator-retractor elastic device 2 disclosed herein can provide a great deal of flexibility in pre-closing the gap 24.

FIG. 4 is a schematic diagram of the tissue portions separated by a gap with the tissue approximator-retractor elastic device of FIG. 1. An operator can provide and position a tissue approximator-retractor elastic device adjacent the tissue portions across the gap 24. In general, one end of the elastic device 2 can be anchored to a first tissue portion 20 with a first tissue engagement member 12 coupled to a first coupling ring 6. The operator can then choose between the remaining coupling rings to select a second coupling ring as suitable for the particular tissue involved, the size of the gap 24, and the amount of contractile force to be exerted by the elastic device 2.

FIG. 5 is a schematic diagram of the two tissue portions separated by the gap with at least two tissue engagement members coupled to the tissue approximator-retractor elastic device. A second tissue engagement member 14 can be inserted into a selected ring, such as ring 10 that is appropriate for the size of the gap 24 and the contractile force to be exerted by the elastic device 2.

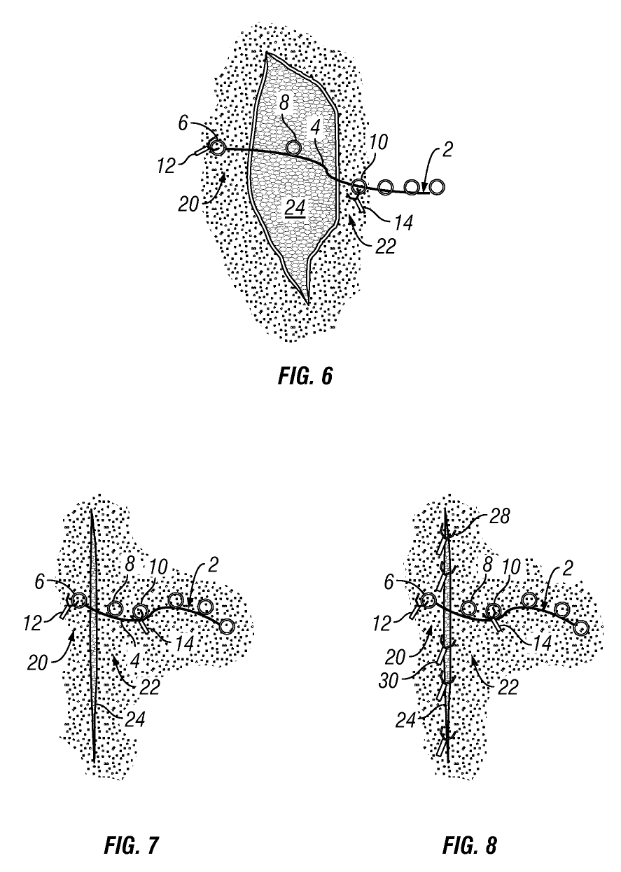

FIG. 6 is a schematic diagram of the tissue portions separated by the gap with the tissue approximator-retractor elastic device anchored on either side of the gap. The elastic portion 4 can be stretched across the gap 24 using the engagement member 14 in conjunction with the ring 10 (or other selected ring) and anchored to the second tissue portion 22. Thus, ring 6 is anchored at the first tissue portion 20 with the first engagement member 12, and the ring 10 is anchored at the second tissue portion 22 with the second engagement member 14. The operator can select another ring, such as ring 8, to be coupled to the second tissue portion 22, if additional contractile force is needed for the gap 24. Alternatively, a ring distal from the ring 6 can be chosen to reduce contractile forces. Such a selection can be made without removing the tissue approximator-retractor elastic device 2 or inserting another elastic device into the area.

FIG. 7 is a schematic diagram of the tissue portions that have been approximated using the tissue approximator-retractor elastic device. The engagement members 12, 14 and their respective rings 6, 10 are anchored to the tissue portions 20, 22, and the elastic portion 4 exerts a contractile (tension) force on the tissue portions. The stretched portion of the elastic portion 4 can contract the gap 24 to a smaller dimension between the first and second tissue portions 20, 22.

FIG. 8 is a schematic diagram of an exemplary closure of the gap between the tissue portions. In a contracted position, the gap 24 can be sufficiently closed so that the additional tissue engagement members 28, 30 can be placed along the gap 24 to secure the closure in the approximated position. Such securing can occur through clips, sutures, and other medical devices known to those with ordinary skill in the art. The tissue approximator-retractor elastic device 2 can be removed if appropriate, or optionally can be allowed to remain for a period of time to help maintain the closure. In general, it is believed that at least one tissue engagement member 28 will be disposed on one side of the tissue approximator-retractor elastic device 2 and at least one tissue engagement member 30 will be disposed on a distal side of the elastic device 2.

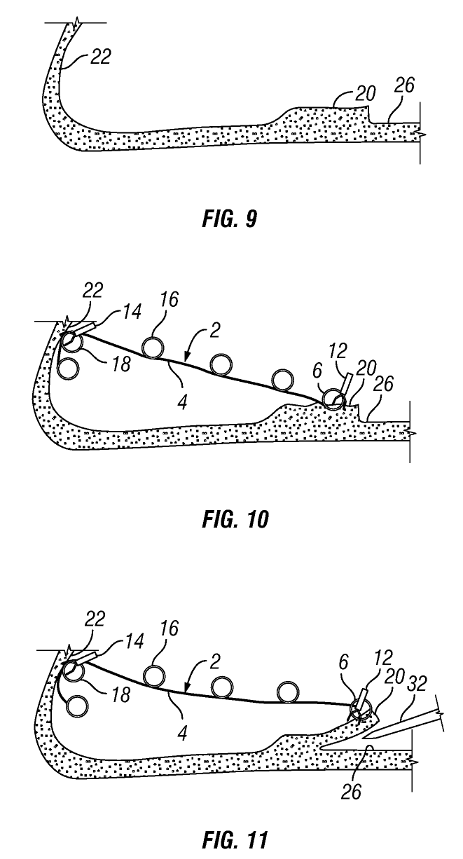

FIGS. 9-14 are schematic diagrams illustrating another exemplary and nonlimiting procedure using the tissue approximator-retractor elastic device. The figures will be described in conjunction with each other.

FIG. 9 is a cross-sectional schematic diagram of tissue having a portion to be excised. A first tissue portion 20, such as tumor, or other growth, is sometimes desired to be excised from a base tissue. When such procedures are done under limited viewing capabilities, such as endoscopically, laparoscopically, or other such restrictive procedures, it is advantageous to have a separate member that can pull the tissue to be excised away from the excision edge to facilitate better viewing and better procedures. The present disclosure can provide such capabilities.

FIG. 10 is a cross-sectional schematic diagram of the tissue of FIG. 9 with a tissue approximator-retractor elastic device coupled thereto. A tissue approximator-retractor elastic device 2 can be provided to the area near the tissue portions 20, 22. For example, a first tissue portion 20 can include the tissue to be excised or otherwise treated. A second tissue portion 22 can be a tissue portion distally located to serve as an anchor point from the area to be excised. The elastic device 2 can include an elastic portion 4 and at least three coupling rings 6, 16, and 18. The first ring 6 can be coupled to the first tissue portion 20 with a first tissue engagement member 12. The elastic portion 4 can be stretched by choosing a remaining coupling ring, such as ring 18, and coupling that ring to the second tissue portion 22 with a second tissue engagement member 14. The contractile force exerted by the elastic portion 4 of the elastic device 2 can create a tension on the first tissue portion 20, so that as it is excised or otherwise treated, it is pulled toward the second tissue portion 22. Thus, the first tissue portion is retracted away from the base tissue and toward the second tissue portion 22.

FIG. 11 is a schematic cross-sectional diagram of the first tissue portion partially excised illustrating a retraction using the tissue approximator-retractor elastic device. A cutter 32 can be used to separate the first tissue portion 20 from a third tissue portion 26, such as the base tissue. The elastic portion of the elastic device 2 is allowed to contract to adjust the location of the first tissue portion 20 relative to the second tissue portion 22 as the first tissue portion is excised. Thus, the area of separation with the cutter 32 can be viewed and more readily accessed in the medical procedure.

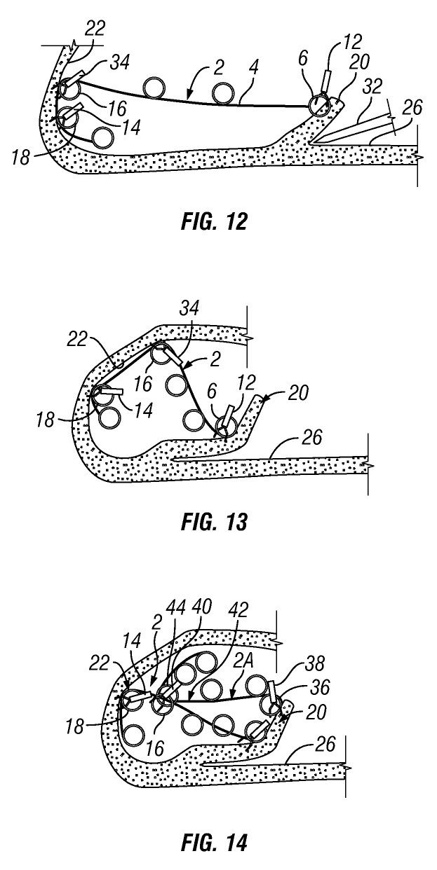

FIG. 12 is a cross-sectional schematic diagram of FIG. 11 illustrating a different coupling of a different ring to the tissue portion compared to the tissue approximator-retractor elastic device. As the separation between the first tissue portion 20 and third tissue portion 26 continues to occur, it may be advantageous to adjust the contractile force of the elastic portion 4 on the first tissue portion 20. In some instances, another ring, such as ring 16, can be coupled to the tissue portion 22 or another tissue portion, with a tissue engagement member 34 to increase the contractile force. Alternatively, the engagement member 14 in the ring 18 can be moved to another location. Further, the ring 18 can be uncoupled from the tissue portion 22 to reduce or eliminate the contractile force.

FIG. 13 is a diagram with a three dimensional perspective illustrating an option to vary an angle of the contractile force between tissue portions. It may be advantageous to change a retraction angle of the first tissue portion 20 during, before, or after the medical procedure. For example, it may be advantageous to concentrate on a certain portion of the first tissue portion 20 and thus retract it at a different angle. The flexible application of the presently disclosed tissue approximator-retractor elastic device 2 with multiple rings allows for a variance in the angle of contraction between the first tissue portion and the second tissue portion, even during the medical procedure. For example, the second ring 18 can be coupled with the second engagement member 14 to the tissue portion 22. During the procedure, it may be advantageous to change the angle of retraction and/or the amount of contraction by coupling a coupling ring 16 with a tissue engagement member 34 to another location of the second tissue portion 22 and at an angle to the second ring 18. In a similar fashion, the angle of approximation can also change when the two tissue partners are approximated.

FIG. 14 is a cross-sectional perspective diagram of the tissue portions to be excised with a plurality of tissue approximator-retractor elastic devices coupled thereto. The flexibility of the current tissue approximator-retractor elastic device 2 with multiple rings can allow attachment of other tissue approximator-retractor elastic devices to assist in the medical procedure. For example, a first tissue approximator-retractor elastic device 2 can be attached to the first tissue portion 20 and the second tissue portion 22, as described above. If desired, a second segment of the first tissue portion 20 can also be coupled to a second tissue approximator-retractor elastic device 2A. A coupling ring 36 on the second tissue approximator elastic device 2A can be coupled to the first tissue portion 20 (or other tissue portion as appropriate) with a tissue engagement member 38. The second tissue approximator-retractor elastic device 2A can be coupled to either a separate tissue portion, similar to coupling the tissue portion with the first elastic device 2, or the elastic device 2A can be coupled to the first tissue approximator-retractor elastic device 2 by use of one of the rings on each of the elastic devices. For example, a ring 44 on the second tissue approximator-retractor elastic device 2A can be coupled with the ring 16 on the elastic device 2. The elastic device 2A in its corresponding elastic portion can be stretched to make such coupling, as appropriate, to apply a contractile force on the tissue portion coupled to the ring 36. Further, if appropriate, the rings 44 and/or 16 can be coupled to another tissue portion 42. The coupling can change the contractile force, the angle of contraction, or other aspects as appropriate.

While the tissues are approximated or retracted, the intended medical procedure can be performed. After the medical procedure that created the desire for the tissue approximation or retraction has been accomplished, the tissue approximator-retractor elastic device or devices can be removed. An incision through which the elastic device was inserted and the medical procedure was performed can be closed in a conventional manner. Alternatively, the elastic device or devices can remain for some time period as might be desired to maintain some amount of tissue approximation or retraction.

The invention has been described in the context of various embodiments and not every embodiment of the invention has been described. Apparent modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, Applicant intends to protect all such modifications and improvements to the full extent that such falls within the scope or range of equivalent of the following claims.

The various methods and embodiments of the invention can be included in combination with each other to produce variations of the disclosed methods and embodiments, as would be understood by those with ordinary skill in the art, given the understanding provided herein. Also, various aspects of the embodiments could be used in conjunction with each other to accomplish the understood goals of the invention. Also, the directions such as “top,” “bottom,” “left,” “right,” “upper,” “lower,” and other directions and orientations are described herein for clarity in reference to the figures and are not to be limiting of the actual device or system or use of the device or system. The term “coupled,” “coupling,” “coupler,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, directly or indirectly with intermediate elements, one or more pieces of members together and can further include without limitation integrally forming one functional member with another in a unity fashion. The coupling can occur in any direction, including rotationally. Unless the context requires otherwise, the word “comprise” or variations such as “comprises” or “comprising”, should be understood to imply the inclusion of at least the stated element or step or group of elements or steps or equivalents thereof, and not the exclusion of a greater numerical quantity or any other element or step or group of elements or steps or equivalents thereof. The device or system may be used in a number of directions and orientations. Further, the order of steps can occur in a variety of sequences unless otherwise specifically limited. The various steps described herein can be combined with other steps, interlineated with the stated steps, and/or split into multiple steps. Additionally, the headings herein are for the convenience of the reader and are not intended to limit the scope of the invention.

Further, any references mentioned in the application for this patent as well as all references listed in the information disclosure originally filed with the application are hereby incorporated by reference in their entirety to the extent such may be deemed essential to support the enabling of the invention. However, to the extent statements might be considered inconsistent with the patenting of the invention, such statements are expressly not meant to be considered as made by the Applicant(s).

1. A method of adjusting a location of a first tissue portion relative to a second tissue portion in a medical procedure, comprising:
providing a tissue approximator-retractor elastic device near the tissue portions, the device having an elastic portion with at least three coupling rings spaced along the elastic portion;
coupling a first coupling ring to the first tissue portion;
choosing between the remaining coupling rings to select a second coupling ring;
engaging the second coupling ring and pulling the elastic portion to the second tissue portion;
coupling the second coupling ring to the second tissue portion; and
allowing the elastic portion of the device to contract to adjust the location of the first tissue portion relative to the second tissue portion.

2. The method of claim 1, further comprising approximating the two tissue portions.

3. The method of claim 2, further comprising securing the two tissue portions while approximating the two tissue portions with the elastic portion.

4. The method of claim 1, wherein the first tissue portion comprises a first wound portion and the second tissue portion comprises a second wound portion, the wound portions being separated by a gap and further comprising:
coupling the first coupling ring to the first wound portion;
coupling the second coupling ring to the second wound portion; and
allowing the elastic portion to approximate the wound portions.

5. The method of claim 1, further comprising retracting the first tissue portion from a third tissue portion by allowing the elastic portion to apply a contractile force between the first tissue portion and the second tissue portion.

6. The method of claim 5, further comprising separating the first tissue portion from the third tissue portion while applying the contractile force between the first tissue portion and the second tissue portion.

7. The method of claim 6, further comprising adjusting the contractile force as the first tissue portion separates from the third tissue portion by coupling to a different coupling ring on the elastic portion.

8. The method of claim 1, further comprising adjusting an angle of contractile force applied between the first tissue portion and the second tissue portion by coupling another coupling ring to another tissue portion.

9. The method of claim 1, further comprising performing the medical procedure internally to an exterior of an organism

10. The method of claim 9, further comprising conducting the medical procedure endoscopically or laparoscopically.

11. The method of claim 1, further comprising coupling a second tissue approximator-retractor elastic device to the first tissue portion and to the first tissue approximator-retractor elastic device.

12. The method of claim 11, further comprising coupling the second tissue approximator-retractor elastic device with the first tissue approximator-retractor elastic device using a different coupling ring than is coupled to the second tissue portion.

13. A tissue approximator-retractor elastic device, comprising:
an elastic portion having a contracted length and adapted to expand to a different length to exert a contractile force; and
at least three coupling rings spaced along the elastic portion and adapted to allow an operator to choose between the rings to couple at least two rings to at least two tissue portions and adjust a contractile force exerted by the elastic portion on the two tissue portions.

14. The device of claim 13, wherein the elastic portion is elongated and the coupling rings are spaced along the elongated elastic portion.

15. The device of claim 13, wherein the coupling rings are spaced along the elastic portion at a spacing of 0.5 cm.

16. The device of claim 13, wherein a third coupling ring is adapted to be coupled to a third tissue portion for further adjusting the relative location between the two tissue portions.

17. The device of claim 13, further comprising a plurality of couplers adapted to be coupled with a plurality of the coupling rings.
